(19) United States
(12) Patent Application Publication
(10) Pub. No.: US 2025/0377356 A1
(43) Pub. Date: Dec. 11, 2025

(54) METHOD FOR MAGNETICALLY DETECTING MICROSCOPIC BIOLOGICAL OBJECTS AND ASSOCIATED DEVICES

(71) Applicant: COMMISSARIAT A L'ENERGIE ATOMIQUE ET AUX ENERGIES ALTERNATIVES, PARIS (FR)

(72) Inventors: Pierre BONVILLE, VERSAILLES (FR); Mathieu JECKELMANN, FRIBOURG (CH); Guénaëlle JASMIN-LEBRAS, GIF-SUR-YVETTE CEDEX (FR); Maïkane DEROO, GIF-SUR-YVETTE CEDEX (FR); Cécile FERAUDET, GIF-SUR-YVETTE CEDEX (FR)

(21) Appl. No.: 18/877,979

(22) PCT Filed: Jun. 16, 2023
(86) PCT No.: PCT/EP2023/066292

§ 371 Date: Feb. 20, 2025

(30) Foreign Application Priority Data
Jun. 24, 2022 (FR) FR2206315

(51) Int. Cl. G01N 33/543; G01N 15/10; G01N 15/1031; G01N 33/569

(52) U.S. Cl. CPC G01N 33/54326; G01N 15/1031; G01N 33/54373; G01N 33/569; G01N 2015/1024

(57) ABSTRACT

A method for determining the magnetic moment of a magnetic object circulating in a microfluidic channel, on which two magnetic field sensors are disposed on either side, the method including receiving two electrical signals; calculating the passage height of the magnetic object into the microfluidic channel from the ratio of the electrical signals and by a reference curve; and calculating the magnetic moment of the magnetic object from the passage height determined and one of the electrical signals.

### TECHNICAL FIELD OF THE INVENTION

The technical field of the invention is the identification of microscopic biological objects from their magnetic moment, with the aim of developing an early and sensitive diagnosis device.

TECHNOLOGICAL BACKGROUND OF THE INVENTION

The development of so-called “early” diagnosis methods and devices that are fast, sensitive, transportable to the patient's bedside and inexpensive is really challenging not only in the healthcare, but also in defence and environment sectors.

Easy-to-use early diagnosis methods include the migration of targets in cellulose, referred to as “strips”. The diagnosis method implementing strips provide results in 30 minutes, but has the drawbacks that it is not adapted to large targets (such as cells and some bacteria) and, above all, that it is not sufficiently sensitive. Other diagnosis methods used in biology laboratories comprise ELISA (Enzyme-Linked ImmunoSorbent Assay) or PCR(Polymerase Chain Reaction) methods. These methods have better sensitivities (count between 10³ CFU/mL and 10⁴ CFU/mL). However, they take between 3 and 4 hours to be performed and require staff trained in their complex use.

A commonly used device for detecting biological objects in biology laboratories is a flow cytometer. It allows biological objects to be counted one by one. However, it is not transportable and is complex to use. It is also expensive.

So-called “labs on chips” or “biochips” provide a solution to the problems of overall size and complexity. For this, some biochips use optical target detection. However, these biochips are not very well adapted to the study of some opaque matrices. Other biochips use electrochemical target detection. However, these biochips have the drawback of having too many non-specific interactions with the external environment or some matrices, which reduces detection sensitivity.

Biochips implementing magnetic detection by means of magnetoresistive sensors have rapidly developed. Biological objects to be detected are marked (also referred to as “labelled”) by means of magnetic particles (or beads) functionalised by antibodies specific to the target of interest. Biochips based on magnetoresistive sensors then detect, one by one, as a function of time, magnetic objects circulating in a microfluidic channel. Detection can be made in liquid matrices, and samples studied do not need to be washed beforehand. They can also be of low concentration. Finally, signal counting and analysis can be performed simultaneously.

Among the magnetic detection methods developed in recent years, magnetoresistive sensors using Giant MagnetoResistance (GMR) or Tunnel MagnetoResistance (TMR) are the only ones to combine high detection sensitivity, small overall size and mature industrial production. These sensors also have low manufacturing and operating costs.

A biochip may comprise a GMR or TMR magnetoresistive sensor disposed beneath the microfluidic channel. Labelled biological objects circulate in said microfluidic channel. The dipole field emitted by each marked biological object passing in proximity to said magnetoresistive sensor is measured. When the dipole field emitted is greater than a limit of detection of the GMR sensor, the signal is counted.

This method, however, comprises some major drawbacks. Indeed, the dipole field is proportional to u/z³, where μ is the magnetic moment of the magnetic object detected and z_{b} its passage height into the channel above the sensor. Thus a signal from a small aggregate of magnetic beads (for example, an aggregate without any biological object) passing close to the GMR sensor (low μ and z) can give the same signal as a properly magnetically marked biological object but passing higher up the channel (larger μ and z). Thus, with this system it is not possible to distinguish between aggregates of magnetic beads and labelled biological objects since it is not possible to determine independently the magnetic moment and the passage height from a single dipole field. The presence of magnetic bead aggregates is due to the fact that it is necessary to place an excess of magnetic beads functionalised by specific antibodies in the liquid matrix to increase the chances of properly labelling biological objects.

Document WO 2019/238857 A1 discloses a magnetic detection method by means of a biochip comprising several pairs of magnetoresistive sensors disposed on either side of the microfluidic channel, especially above and below the microfluidic channel. The magnetoresistive sensors in a pair are perfectly aligned on either side of the channel. A marked biological object passing between both sensors of a same pair will therefore be simultaneously detected by these two sensors. The two signals simultaneously emitted by two sensors in the same pair (for example on the channel referred to as “TOP” signal and under the channel referred to as “BOTTOM” signal) are said to be “synchronised”.

The method disclosed likewise provides for determining the trajectory followed by the object from the ratio of the amplitudes of signals measured by successive pairs of sensors arranged along the microfluidic channel.

There is therefore a need to determine the height and magnetic moment of a marked biological object from the ratio of the amplitudes of signals.

### SUMMARY OF THE INVENTION

In this context, the invention relates to a method for determining the magnetic moment of a magnetic object by means of a biochip, the biochip comprising: a microfluidic channel extending in a plane and having a height, measured along a “normal direction” perpendicular to the plane; and two magnetic field sensors, disposed on either side of the microfluidic channel, the method comprising the steps of:
receiving two synchronised electrical signals from the magnetic field respectively, both electrical signals corresponding to the passage of the magnetic object into the microfluidic channel at each magnetic field sensor;
calculating the passage height of the magnetic object, measured along the normal direction, from the ratio of the amplitudes of the synchronised electrical signals and by means of a reference curve relating the passage height of a calibration magnetic object into the microfluidic channel to a ratio of the amplitudes of the calibration electrical signals corresponding to the passage of the calibration magnetic object at said passage height, the ratio of the amplitudes of the synchronised electrical signals being equal to the ratio of the amplitudes of the calibration electrical signals;
calculating the magnetic moment of the magnetic object from the passage height determined and one of the synchronised electrical signals.

By synchronised electrical signals, it is meant that the electrical signals correspond to the measurement of the same magnetic object, even if the sensors are not aligned (the electrical signals may then show a time shift between them, for example proportional to the speed of movement of the magnetic object in the microfluidic channel).

By virtue of analysing simultaneous (synchronised) signals and determining the passage height, it is possible to determine the magnetic moment of the object detected, which is either a biological object marked with magnetic beads, an aggregate of magnetic beads or magnetosomes

Calculating the passage height, through the use of a calibrated reference curve based on a calibration magnetic object, enables passage height of the magnetic object to be reliably determined and therefore the calculation of its magnetic moment.

As a result, the discrimination of magnetic objects to be detected is improved and makes it possible to discriminate biological objects marked with a large number of magnetic beads relative to aggregates mainly consisting solely of magnetic beads (and comprising no magnetic object). False positives related to aggregates can thus be distinguished from true positives related to marked biological objects.

FIG. 1 shows an example of a reference curve relating the ratio R of the amplitudes of calibration electrical signals as a function of the passage height z_{b} of a calibration magnetic object having radius Ro, into the microfluidic channel having height hcan. The passage height z_{b} can therefore only vary between Ro and hcan-Ro. The ratio is represented by the continuous curve and noted “R”. Said ratio R assumes a minimum value when the passage height z_{b} of the calibration object is minimum (when z_{b}=Ro). The ratio R non-linearly increases as the passage height z_{b} of the calibration magnetic object increases until it reaches a maximum value (when z_{b}=hcan−Ro). [FIG. 1] also shows a ratio RAA, in dashed line, calculated according to prior art and especially according to the teachings of document WO 2019/238857 A, the calculation being adapted to the present parameter values. The ratio RAA is linear with the passage height z_{b} and passes through 1 when the magnetic object under consideration is equidistant from the magnetic field sensors. It is therefore observed that both ratios R and RAA are only equal for two points; z_{b}=0 and z_{b}=z_{bm}. The figure also shows an absolute value difference E (dash-dotted curve) between both ratios R and RAA, which can be equated with an absolute error. The difference E varies as a function of the passage height z_{b} and can take on significant values in some cases (especially around z_{bm}/2 or when z_{b} is greater than z_{bm}, where R tends towards infinity).

The reference curve therefore makes it possible to improve estimation of the passage height of the magnetic object and thus improve determination of its magnetic moment.

It should be added that the preliminary determination of the passage height serves not only to reduce a systematic error. Indeed, once the passage height has been determined, it is possible to determine a theoretical signal of a bead whose magnetic moment is known from preliminary measurements with a magnetometer. The ratio of the experimental signal to the theoretical signal of a bead allows us to determine the number of beads N contained by the object detected (originating the experimental signal) and consequently the magnetic moment which is then N times the magnetic moment of a bead.

The method is also compatible with a biochip of prior art (such as that disclosed in WO 2019-238857 A).

The method can also be used to characterise aggregates of magnetic beads (not comprising a biological object) developed for different applications. Indeed, macroscopic magnetisation measurement techniques, such as vibrating sample magnetometers or a “SQUID” magnetometer, do not allow the magnetic moment of single micrometric samples to be measured.

By disposed on either side of the microfluidic channel, it is meant disposed on either side of the height of the microfluidic channel.

Advantageously, the magnetic field sensors are disposed opposite each other and aligned along the normal direction.

Advantageously, the reference curve is determined from the calibration magnetic object having a calibration magnetic moment independent of the magnetic moment of the magnetic object. Thus, the reference curve can be established regardless of the final magnetic objects to be characterised.

Advantageously, the electrical signals from the magnetic field sensors are proportional to the dipole fields emitted by the magnetic object and perceived by the magnetic field sensors. In other words, the ratio of the amplitudes of the electrical characterisation signals is equal to the ratio of the amplitudes of the dipole field emitted by the calibration magnetic object on each of the magnetic field sensors.

Advantageously, the electrical signal from each magnetic field sensor is equal to the dipole field emitted by the magnetic object multiplied by a sensitivity factor of the magnetic field sensor, the sensitivity factor of the magnetic field sensor being advantageously linear and preferably equal to 2%/mT, or even 1%/mT. Advantageously, the sensitivity factors of both magnetic field sensors are identical.

Even more advantageously, each calibration electrical signal is equal to the dipole field emitted by the calibration magnetic object at a calibration magnetic field sensor multiplied by a calibration sensitivity factor, the calibration sensitivity factors being equal to the sensitivity factors of both magnetic field sensors.

Advantageously, the method comprises a step of determining the reference curve comprising the following sub-steps of:
for different passage heights of the calibration magnetic object between two calibration magnetic field sensors:
   determining the dipole magnetic field emitted by the calibration magnetic object and perceived by one of the calibration magnetic field sensors;
   determining the dipole magnetic field emitted by the calibration magnetic object and perceived by the other of the calibration magnetic field sensors;
calculating the ratio of the amplitudes of the dipole magnetic fields perceived by the calibration magnetic field sensors as a function of the different passage heights of the calibration magnetic object.

Advantageously, the reception step also comprises a sub-step of identifying synchronised electrical signals comprising measuring a time difference between the characteristic signatures of both electrical signals, synchronisation being identified when the time difference is within a predetermined time range, the predetermined time range preferably being determined from a speed of movement of the magnetic object. Each characteristic signature corresponds to the measurement of the magnetic object by one of the magnetic field sensors.

Advantageously, the amplitudes of the electrical signals are preferably estimated at the characteristic signatures of each electrical signal.

Advantageously, the reception step comprises a sub-step of identifying characteristic signatures in each of the electrical signals from shape criteria of the electrical signals.

Advantageously, the magnetic object is a biological object marked by means of magnetic beads.

Advantageously, the method also comprises a step of calculating the number of magnetic beads associated with the biological object marked from the magnetic moment calculated and the magnetic moment of a single magnetic bead. The magnetic moment of a single magnetic bead can be deduced from a magnetic moment measurement of an assembly of magnetic beads, the magnetic moment measurement being performed, for example, by means of a vibrating sample magnetometer.

Advantageously, the magnetic field sensors are magnetoresistive sensors and are preferably based on the giant magnetoresistance (GMR) effect or the tunnel magnetoresistance (TMR) effect.

The invention also relates to a device for determining the magnetic moment of a magnetic object, comprising a biochip and means adapted to implement the method for determining the magnetic moment of a magnetic object according to the invention.

The invention also relates to a computer program comprising instructions which cause the aforementioned device to execute the steps of the method for determining the magnetic moment of a magnetic object according to the invention.

The invention further relates to a computer-readable medium having the aforementioned computer program recorded thereon.

The invention also relates to a method for counting biological objects comprising the following steps of:
marking the biological objects by mixing them with a liquid matrix comprising magnetic beads able to attach to receptors carried by each biological object;
circulating the liquid matrix comprising the biological objects marked into the microfluidic channel of the biochip and determining the magnetic moment of magnetic objects passing between the magnetic field sensors by means of the method for determining the magnetic moment according to the invention, each magnetic object counting as a biological object marked when the magnetic moment associated therewith is greater than a threshold magnetic moment.

The counting method thus makes it possible to count the biological objects marked. For example, it enables a histogram of the passage heights of the different magnetic objects detected to be obtained. It also enables a histogram of the magnetic moments (and therefore the number of magnetic beads) associated with the magnetic objects detected to be obtained. The counting method can also allow counting of biological objects simultaneously with circulating the liquid matrix comprising the biological objects marked in the microfluidic channel of the biochip.

Advantageously, the counting method comprises the step of determining the threshold magnetic moment, said step comprising the following sub-steps of:
circulating the liquid matrix without biological objects, referred to as the “negative control”, into the microfluidic channel of the biochip and determining the magnetic moment of magnetic objects passing between the magnetic field sensors by means of the method for determining magnetic moment according to the invention; and
setting the threshold magnetic moment from a statistical distribution of the magnetic moments determined.

The invention also relates to a system for counting biological objects, comprising a biochip and means adapted to implement the counting method of the invention.

The invention also relates to a computer program comprising instructions which cause the counting device according to the invention to execute the steps of the method of the invention.

Finally, the invention further relates to a computer-readable medium having the aforementioned computer program recorded thereon.

The invention and its different applications will be better understood upon reading the following description and upon examining the accompanying figures.

### BRIEF DESCRIPTION OF THE FIGURES

The figures are set forth by way of indicating and in no way limiting purposes of the invention. Unless otherwise specified, a same element appearing in different figures has a single reference.

FIG. 1 shows an example of a reference curve implemented by the determination method of the invention.

FIG. 2 schematically represents a biochip that can be implemented by the determination method of the invention.

FIG. 3 schematically represents an example of a stack of layers for obtaining a magnetic field sensor as used in the biochip of FIG. 2.

FIG. 4 shows an example of the application of a magnetic field to a stack of layers as illustrated in FIG. 3.

FIG. 5 represents, in a top view, an example of the geometry of a magnetic field sensor as used in the biochip of FIG. 2.

FIG. 6 represents an example of the arrangement of magnetic field sensors within a biochip as illustrated in FIG. 2.

FIG. 7 represents a first example of electrical signals from a biochip as illustrated in FIG. 2 when a magnetic object passes between its two magnetic field sensors.

FIG. 8 represents a second example of electrical signals from a biochip as illustrated in FIG. 2 when a magnetic object passes between its two magnetic field sensors.

FIG. 9 represents a third example of electrical signals from a biochip as illustrated in FIG. 2 when a magnetic object passes between its two magnetic field sensors.

FIG. 10 schematically represents a mode of implementation of the determination method of the invention.

FIG. 11 represents a mode of implementation of a step of the method of FIG. 10.

FIG. 12 represents an example of a dipole field calculated at different positions and for different numbers of magnetic beads.

FIG. 13 schematically represents an embodiment of a determination device according to the invention.

FIG. 14 represents an example of the acquisition chain for the device of FIG. 13.

FIG. 15 schematically illustrates a mode of implementation of a counting method of the invention.

FIG. 16 schematically represents the principle of marking a biological object so that it can be counted using the method of FIG. 15.

FIG. 17 represents an example of a biological object 5 marked.

FIG. 18 represents an example of a result obtained by means of the counting method of FIG. 15.

### DETAILED DESCRIPTION

FIG. 2 schematically represents a biochip 3 that can be implemented by a method 1 according to the invention. Prior document WO 2019-238857 A describes another embodiment of a biochip 3 that can be implemented by method 1. The biochip 3, in the example of FIG. 2, comprises a microfluidic channel 31 that extends in a plane P. The figure shows a portion of this microfluidic channel 31 which additionally extends along a direction Y, parallel to the plane P. Advantageously, the channel has an inlet and an outlet so that a fluid to be analysed can be circulated, for example along the direction Y, or even in the direction of increasing Ys. The microfluidic channel 31 may have a rectangular cross-section, as illustrated herein, or a different cross-section, provided that it allows magnetic field sensors to be arranged. The cross-section of the microfluidic channel 3 has, for example, a height hcan, measured along a direction Z (referred to as the “normal direction”) perpendicular to the plane P, of between 20 μm and 25 μm. It may have a width L, measured along a direction X perpendicular to the directions Y and Z, of between 100 μm and 150 μm.

Biochip 3 also comprises first and second magnetic field sensors 31, 32. The sensors 32, 33 are disposed on either side of the microfluidic channel 31. The first sensor 32, also referred to as the “top” or “upper” sensor, is disposed on the channel 31, for example. A second sensor 33, also referred to as the “bottom” or “lower” sensor, is disposed under the channel 31, for example. In the embodiment of [FIG. 2], the sensors are disposed opposite each other. They are especially aligned, one above the other along the normal direction Z. By “opposite”, it is meant that the top and bottom sensors 31, 32 each have one orientation and are oriented facing each other. In other words, each sensor 31, 32 may comprise a sensitive part 321, 331, for performing magnetic field measurement. These two sensitive parts 321, 331 can then be oriented facing each other. The sensitive parts 321, 331 are additionally advantageously oriented towards the microfluidic channel 3, or even in contact therewith.

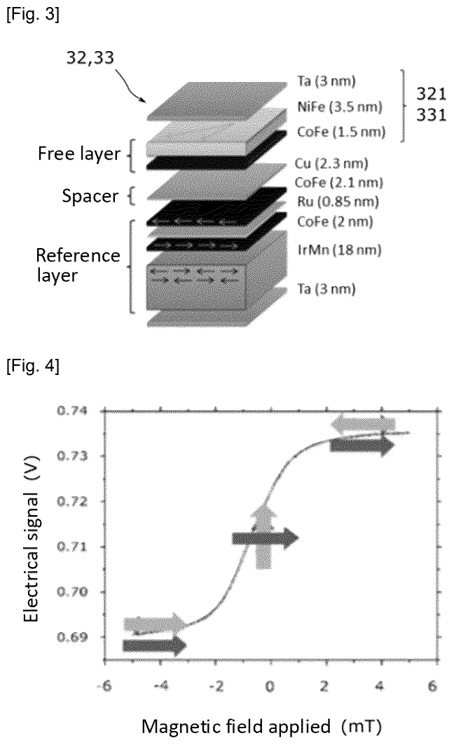

FIG. 3 shows an example of a stack of layers for obtaining a magnetic field sensor 32, 33. This is herein a magnetoresistive sensor. It could also be a so-called “SQUID” sensor implementing a superconducting loop. The layers selected in this example make it possible to obtain a giant magnetoresistance (GMR) effect. They could also be selected to obtain a Tunnel MagnetoResistance (TMR) effect. In both cases (and especially when used as a sensor) the resistance of the stack varies as a function of the orientation of magnetisation of a so-called “free” layer, relative to the orientation of magnetisation of a so-called “reference” layer.

For example, [FIG. 4] shows the effect of applying a magnetic field to a stack of layers as illustrated in [FIG. 3]. The magnetic field is applied antiparallel to the orientation of magnetisation of the reference layer (magnetisation of the reference layer is considered fixed). When both magnetisations of the free and reference layers are parallel, the resistance of the stack is low. The voltage measurable thereacross, for a given polarisation current, is therefore low. Conversely, when both magnetisations of the free and reference layers are antiparallel, the stack resistance is high. The voltage measurable thereacross is therefore high.

There is a range in which magnetisations of the free and reference layers are neither parallel nor antiparallel, but form an acute angle. In this range, the resistivity linearly varies with the angle between both magnetisations. The slope, for example in %/mT (considering the variation rate of the resistance) or in Ω/mT or even in V/mT (when considering the voltage across the stack for a predetermined current), in this range therefore corresponds to the sensitivity of the stack. It is this range which is preferentially considered for making the top and bottom sensors 32, 33 since it enables small variations in the orientation of magnetisation of the free layer to be measured, for example, when the latter is disturbed by a magnetic object such as a biological object marked.

The free layer advantageously forms the sensitive part 321, 331 of the sensors 32, 33.

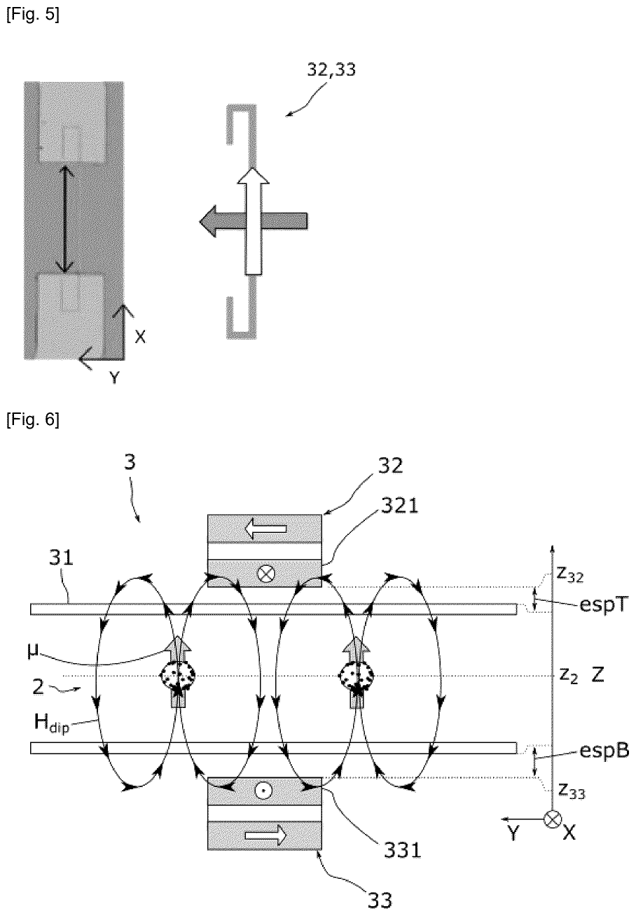

FIG. 3 and FIG. 5 show an example of stack of layers and the geometry of the stack for obtaining magnetoresistive sensors 32, 33 capable of operating in the linear range as described. The stack set forth in FIG. 3 comprises two ferromagnetic layers spaced apart by a non-magnetic material referred to as a “spacer”. In the case of a stack implementing the GMR effect, as of FIG. 3, the spacer is a layer of a non-magnetic metal such as copper. In the case of a stack implementing the TMR effect, the spacer is a dielectric layer, for example of MgO. The so-called “reference” ferromagnetic layer, has a fixed net magnetisation on one of its faces (i.e. its orientation and amplitude are constant). The reference layer comprises, for example, two ferromagnetic sublayers, coupled in an antiferromagnetic manner (for example by exchange coupling by means of the 0.85 nm thick Ru sublayer) so as to form an apparently antiferromagnetic sublayer, referred to as the “synthetic antiferromagnetic” or “SyAF” sublayer. The SyAF sublayer then has a top sublayer with a fixed net magnetisation. The reference layer may also comprise an antiferromagnetic (natural, as opposed to synthetic) sublayer of IrMn, for enhancing magnetisation of the SyAF sublayer.

The so-called “free” ferromagnetic layer has free magnetisation. For this, this layer can be made from a soft magnetic material such as CoFe or NiFe, or from a stack of sublayers of the CoFe/NiFe type.

The stack is advantageously disposed between two metal electrodes, Ta for example, for circulating an electric current therewithin.

Magnetisations of the free and reference layers are preferably parallel to the plane of the layers. The orientation of magnetisation of the reference layer is imposed, for example by the IrMn layer. The zero-field orientation of magnetisation of the free layer can be imposed by another means, for example by means of shape anisotropy. For this, the free layer advantageously has an elongate shape along a direction in the plane of the layers. In this way, magnetisation of the free layer will spontaneously orient itself along this direction. In the embodiment of [FIG. 5], the free layer has a yoke shape (also referred to as a “C” or “horseshoe” shape) with a long, narrow segment located at the stack. This yoke shape allows single-domain magnetisation of the free layer. The long, narrow segment also creates shape anisotropy that will orient magnetisation of the free layer along the great length (herein along axis X). The orientation of the great length of the segment is advantageously selected perpendicular to the orientation of magnetisation of the reference layer (the latter being herein oriented along the axis Y). In this way, magnetisations of the free and reference layers are, in the absence of disturbance, perpendicular to each other. In this way, the magnetoresistive sensors 32, 33 operate in the linear range, where sensitivity is maximum and constant.

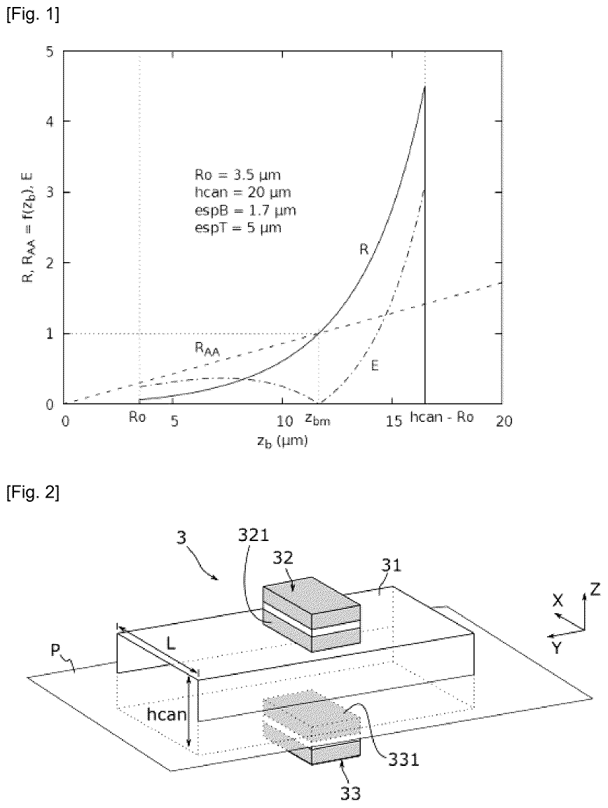

FIG. 6 shows an example of arrangement of the magnetic field sensors 32, 33, herein magnetoresistive, on either side of the microfluidic channel 31. In this example, the sensors 32, 33 are aligned along the direction Z. The sensitive parts 321, 331, herein comprising the free layers of the sensors 32, 33, are oriented facing the channel 31. They are also advantageously disposed in proximity to the microfluidic channel 3. The sensitive parts 321, 331 may be in contact with the microfluidic channel 31. However, this case may be difficult to perform in practice because technological limits, for example relating to the steps of manufacturing the biochip 3, may require separation between the sensors 32, 33 and the channel 31. The top sensor 32 is for example separated from the channel 31 by a distance espT from the channel 31, for example between 3 μm and 7 μm (in the example in [FIG. 1] the value of 5 μm is considered). This distance corresponds, for example, to a layer of material separating the channel 31 from the top sensor 32 and necessary for making the channel 31 and/or the top sensor 32. Likewise, the bottom sensor 33 is for example separated from the channel 31 by a distance espB, for example between 0.3 μm and 3 μm, for example equal to 1.7 μm.

By height, it is meant a distance measured along the normal direction Z. This nomenclature should not be taken literally, however, as the technical effects are invariant to rotation about direction Y. In other words, the sensors 32, 33 could be disposed on the sides of channel 31 rather than on the top and bottom of channel 31. The height thus becomes a width. To simplify the description, however, only examples that take height into account are considered below.

In [FIG. 6], magnetisations of the free layers of sensors 32, 33 are antiparallel to each other and oriented along the direction X (especially transverse to the flow direction Y). Magnetisations of the reference layers of the sensors 32, 33 are also antiparallel to each other and oriented in parallel to the flow direction Y.

FIG. 6 shows a magnetic object 2 at two different positions in the microfluidic channel 3. The magnetic object 2 follows, for example, the flow of a fluid matrix in the channel 3, the flow being in the direction of increasing Ys, for example. The object is therefore first to the right and then to the left, a short time later. The magnetic object 2 follows the flow at a constant height z, measured along the normal direction Z.

The magnetic object 2 is, for example, a marked biological object (such as a molecule or a cell), a magnetosome (a molecule with an intrinsic magnetic moment) or even an aggregate of magnetic beads. By “magnetic beads” it is intended microparticles or nanoparticles to be attached to a biological object. In [FIG. 6], a marked biological object is considered. The magnetic object 2 carries a dipole moment u. FIG. 6 illustrates the orientation of the dipole moment u, which is substantially oriented along the direction Z. The dipole moment μ of magnetic object 2 is due to the functionalisation of a biological object with magnetic beads (illustrated by the small black dots on the white circle). This is referred to as a marked biological object (marking principle illustrated in FIG. 8).

The component along the axis Y of the magnetic field H_{dip} emitted by the magnetic object 2 is perceived by the top 32 and bottom 33 sensors. FIG. 6, shows two field lines H_{dip} emanating from magnetic object 2 as well as the direction of the field H_{dip}. According to whether the magnetic object 2 is to the right or left of sensors 32, 33 (i.e. upstream or downstream of the sensors), the lobe of the field lines coupled to sensors 32, 33 is not the same. Nor is the perceived magnetic field oriented in the same direction for both sensors 32, 33.

When the magnetic object 2 is:
to the right (in the figure) of sensors 32, 33, i.e. upstream (along the flow direction Y), then:
   the lobe of the radiated magnetic field (herein the one on the left) tends to orient magnetisation of the free layer of the top sensor 32 to the left; and
   the magnetisation of the free layer of the bottom sensor 33 to the right;
to the left (in the figure) of sensors 32, 33, i.e. downstream (along the flow direction Y), then:
   the lobe of the radiated magnetic field (herein the one on the right) tends to orient magnetisation of the free layer of the top sensor 32 to the right; and
   the magnetisation of the free layer of the bottom sensor 33 on the left.

The effect of the change in lobe coupled to the free layers of sensors 32, 33 when the magnetic object 2 passes is perceived (and therefore measurable).

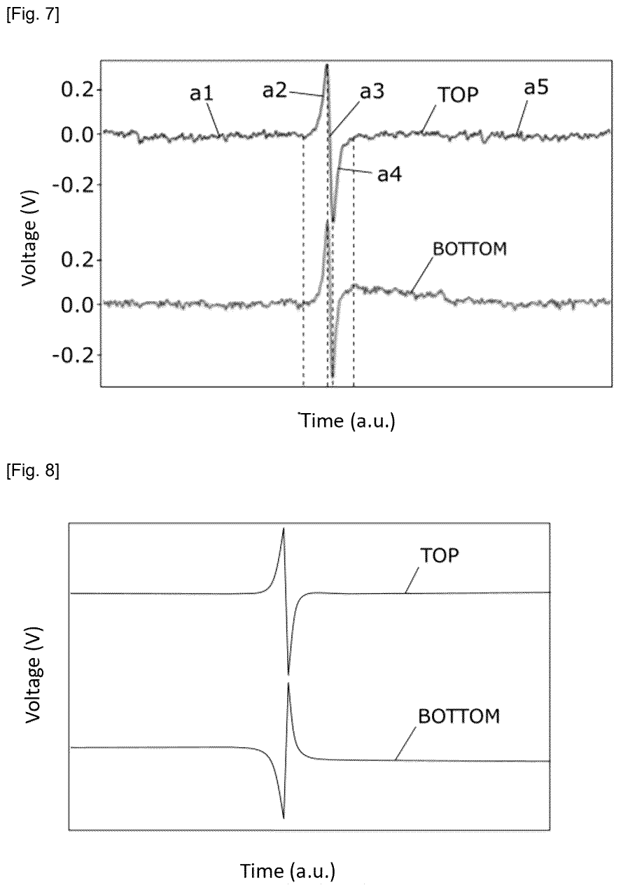

FIG. 7 illustrates an example of the electrical signals emitted by both sensors 32, 33 as a function of time, when a magnetic object 2 passes between both sensors 32, 33. The TOP curve represents, for example, the electrical signal emitted by the top sensor 32 and the BOTTOM curve represents, for example, the signal emitted by the bottom sensor 33. Each signal shows a characteristic signature of the measurement of a magnetic object 2. This is herein a first voltage peak immediately followed by a second peak whose polarity is reversed.

The particular shape of this signature can be explained as follows. At point a1, the TOP curve shows zero voltage, indicating that no magnetic object is being measured. The magnetic object 2 is upstream of the sensors 32, 33 and its radiated field is still undetectable. At point a2, a non-zero electrical signal shows that magnetisation of the free layer of the top sensor 32 is disturbed by a magnetic object 2. A first lobe of the radiated field of the magnetic object therefore influences magnetisation of the free layer. The object is therefore between sensors 32, 33 and slightly upstream of the sensors 32, 33. At point a4, the non-zero electrical signal shows that magnetisation of the free layer of the top sensor 32 is also disturbed by a magnetic object 2. However, the signal is negative, indicating that a second lobe of the radiated field of the magnetic object influences magnetisation of the free layer along an opposite direction. The object is therefore between the sensors 32, 33 and slightly downstream of the sensors 32, 33. At point a3, the signal is zero, indicating that magnetisation of the free layer has returned to its equilibrium direction. The magnetic object is disposed between both sensors 32, 33 and is exactly centred (along the flow direction) relative to the sensors 32, 33. The two lobes of the magnetic object's radiated field influence magnetisation of the layer equally. At point a5, the magnetic object is downstream of sensors 32, 33 but is disposed too far from the sensors for its field to be felt.

The extent of a characteristic signature solely depends on the speed at which the object passes at the sensors. It therefore depends mainly on the flow rate in the microfluidic channel 31.

In this example, the TOP and BOTTOM curves have virtually identical characteristic signatures. When the TOP curve is positive, so is the BOTTOM curve. The maxima and minima of the TOP and BOTTOM curves appear at the same times and reach comparable values. This indicates that the object is simultaneously detected by both sensors (remember that in this example, the sensors are aligned with each other).

When the sensors 32, 33 are aligned along a direction transverse to the flow, herein along the direction Z, the passage of the magnetic object 2 between both sensors 32, 33 triggers a simultaneous response having similar shape but whose amplitude may depend on the passage height z₂ of the object 2.

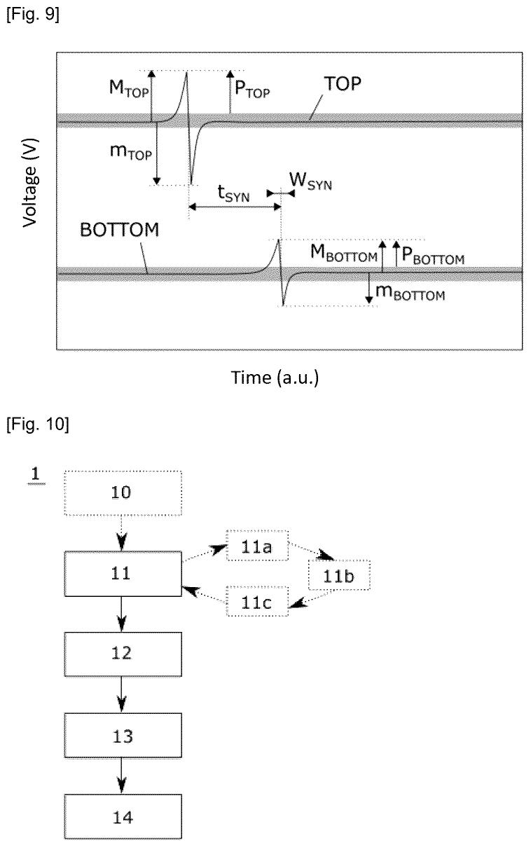

The response to a same magnetic object can however vary in two different ways, illustrated in FIG. 8 and FIG. 9.

For example, in FIG. 8, the polarity of the characteristic signature of the BOTTOM curve is reversed relative to that of the TOP curve. On the other hand, the characteristic signatures are simultaneous. This is due, for example, to the fact that the orientation of magnetisation of the free layer of the second sensor 33 is reversed relative to the orientation of magnetisation of the reference layer of the second sensor 33.

In FIG. 9, the BOTTOM curve has a smaller maximum amplitude than the TOP curve. However, the polarities of the signatures are identical. The magnetic object 2 therefore passed closer to the top sensor 32 than to the bottom sensor 33. The characteristic signatures of both curves do not occur at the same times. The signature of the TOP curve occurs before that of the BOTTOM curve. This may be because the field sensors 32, 33 are not aligned along the direction Z (contrary to what is shown in [FIG. 6]. The top sensor 32 (from which the TOP curve is derived) is placed upstream of the bottom sensor 33 (from which the BOTTOM curve is derived) relative to the flow direction in the microfluidic channel 31. Hence, the magnetic object 2 first disturbs the top sensor 32 and then the bottom sensor 33. The time offset t_{SYN} between these two signatures (measured at the centre of the signatures, when the signal is zero) depends on the speed at which the magnetic object 2 passes at the sensors 32, 33. The time offset t_{SYN} therefore depends on the flow rate in the microfluidic channel 31.

FIG. 6 shows, along the normal direction Z, the passage height z₂ of the magnetic object 2 into the microfluidic channel 3. This passage height z₂ is to be taken into consideration relative to the respective heights z₃₂, z₃₃ of the top 32 and bottom 33 sensors. The heights z₃₂, z₃₃ taken into account are additionally preferably the heights, along the normal direction Z, of the free layers of each sensor.

When the passage height z₂ is equidistant from the heights z₃₂ (of the top sensor) and z₃₃ (of the bottom sensor), the signals measured have the same amplitudes (as illustrated in FIG. 7 and FIG. 8). When the passage height z₂ of the magnetic object 2 is not equidistant, i.e. the magnetic object 2 passes closer to the top sensor 32 or the bottom sensor 33, the signals measured have different amplitudes (as illustrated in FIG. 9).

The method 1 according to the invention makes it possible to detect passage of a magnetic object 2 between both sensors 32, 33 by resorting to determining its magnetic moment u. For this, method 1 provides precise determination of the passage height z₂ of the magnetic object between both sensors 32, 33. This is because the field radiated at the top sensor 32 is proportional to μ/(z₂-z₃₂)³. A good estimate of the passage height therefore makes it possible to estimate magnetic moment of the magnetic object 2.

Method 1 can thus be applied to detecting or counting magnetic objects or even to sorting magnetic objects as a function of their magnetic moment.

FIG. 10 schematically represents a mode of implementation of method 1. According to one step, method 1 provides receiving 11 the electrical signals from the top 32 and bottom 33 sensors respectively. These are, for example, the electrical signals illustrated by the TOP and BOTTOM curves in [FIG. 9]. These electrical signals correspond to the passage of the magnetic object 2 into the microfluidic channel 31 at a passage height z₂ which has to be determined. The signals received are particular in that they correspond to the passage of the same object 2. They are moreover said to be “synchronised”. For example, they have characteristic signatures of the measurement of a same magnetic object 2 and the difference between these characteristic signatures coincides, for example, with the flow rate in the microfluidic channel 31.

Method 1 comprises a step 12 of calculating the passage height z₂ of the magnetic object 2 from the previously received synchronised electrical signals. For this, method 1 provides for the use of a reference curve R(z_{b}) to obtain the height z₂ of the magnetic object 2 in channel 3 from the ratio of the amplitudes of the synchronised electrical signals.

To apply this to the synchronised signals in FIG. 9, the amplitude ratio is obtained, for example, from the maximum positive amplitudes M_{TOP}, M_{BOTTOM} of the TOP, BOTTOM synchronised signals, by calculating, for example, M_{TOP}/M_{BOTTOM}. The ratio of amplitudes could also be obtained from the maximum negative amplitudes M_{TOP}, M_{BOTTOM} of the synchronised TOP, BOTTOM signals, by calculating m_{TOP}/m_{(BOTTOM)}, for example. It is also contemplatable to calculate a linear combination of both aforementioned ratios a×M_{TOP}/M_{BOTTOM}+b×m_{TOP}/m_{BOTTOM}.

The ratio of the amplitudes obtained is compared with the reference curve R(z_{b}), as illustrated in FIG. 1. FIG. 1 illustrates an example of the reference curve R(z_{b}). The curve R relates the passage height z_{b} of a calibration magnetic object into the microfluidic channel 31 as a function of a ratio R of amplitudes of calibration electrical signals. A direct reading of the curve provides a passage height (noted z_{b}) of the calibration magnetic object in FIG. 1 that is equal to the passage height z₂ of the magnetic object 2 targeted (in other words, z₂=z_{b}).

The method 1 according to a third step provides calculating 13 the magnetic moment μ of the magnetic object 2. For this, the passage height z₂ previously determined is used. It is compared with the first electrical signal and/or the second electrical signal. Indeed, each electrical signal is proportional to the dipole magnetic field perceived at the sensors 32, 33 which, in turn, depend on the distance of the magnetic object 2 relative to the sensors 32, 33 and its magnetic moment μ.

For example, one way of doing this is to consider the electrical signal V₃₂ measured by the top sensor 32. It can be approximated by

$${V}_{23} \approx {S}_{32} \cdot μ / {r}^{3}$$

where S₃₂ is the sensitivity of the top sensor 32, μ is the magnetic moment of the target object 2 and r is the distance of the target object 2 relative to r. When the voltage measured across the first sensor is maximal (for example M_{TOP} in FIG. 9), the magnetic object 2 is positioned under the top sensor 32. It is not exactly in vertical alignment with the top sensor 32 (since it has been seen that the measured signal there is zero, as discussed with reference to FIG. 7) but its distance r from the first sensor 32 is very close to the passage height z₂. The voltage V₃₂ can therefore be estimated by

$${V}_{32} = {S}_{32} \cdot μ \cdot {z}_{2}$$

The magnetic moment μ of magnetic object 2 is therefore obtained by calculating

$$μ = \frac{{V}_{32}}{{S}_{32} \cdot {Z}_{2}}$$

This first way of doing this makes it possible to determine the magnetic moment of the magnetic object 2. The determination accuracy does not herein depend on the relative position of the object to the sensors 32, 33.

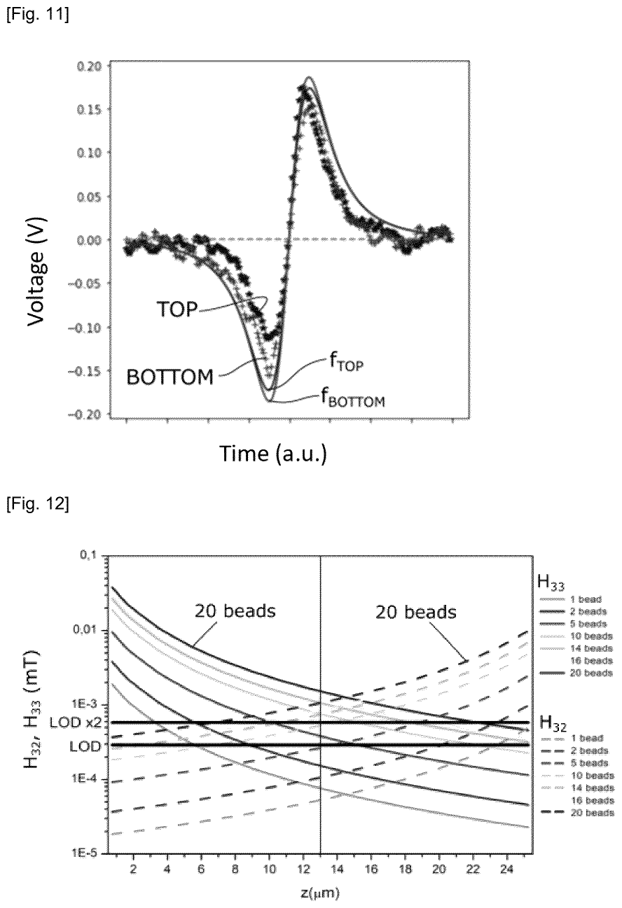

FIG. 11 shows another way of performing calculation 13 of the magnetic moment μ of the magnetic object 2. In this calculation, the electrical signals TOP, BOTTOM measured across both sensors 32, 33 are taken into account. Two theoretical curves f_{TOP}, f_{BOTTOM} are fitted to both TOP, BOTTOM signals. The time extent of the characteristic signatures largely depends on the speed of passage of the magnetic object 2 between both sensors 32, 33 and therefore on the flow rate, which is known. On the other hand, the amplitude of the TOP and BOTTOM curves at any instant depends on the magnetic moment μ carried by the magnetic object 2. Fitting the theoretical curves f_{TOP}, f_{BOTTOM} therefore depends solely on the magnetic moment u of the magnetic object 2. The determination of the magnetic moment μ can be further improved by taking the values measured by both sensors 32, 33 into account, as well as by fitting the measured values.

The theoretical curves f_{TOP}, f_{BOTTOM} take account of an estimate of the magnetic field H radiated by the magnetic object 2 and perceived by a sensor. For example, the field H₃₂ perceived by the top sensor 32 and radiated by a magnetic object 2 at the coordinates x₂, y₂ and z₂ is:

$${H}_{32} = ( \frac{{y}_{l}}{{q}_{2}^{2}} ( \frac{{x}_{r}}{{r}_{2}} - \frac{{x}_{l}}{{r}_{1}} ) + \frac{{y}_{r}}{{q}_{4}^{2}} ( \frac{{x}_{l}}{{r}_{4}} - \frac{{x}_{r}}{{r}_{3}} ) ) sin θsin ψ + ( \frac{1}{{r}_{1}} - \frac{1}{{r}_{2}} + \frac{1}{{r}_{3}} - \frac{1}{{r}_{4}} ) sin θcos ψ + ( \frac{h}{{q}_{2}^{2}} ( \frac{{x}_{l}}{{r}_{1}} - \frac{{x}_{r}}{{r}_{2}} ) + \frac{h}{{q}_{4}^{2}} ( \frac{{x}_{r}}{{r}_{3}} - \frac{{x}_{l}}{{r}_{4}} ) ) cos θ )$$

$$with :$$

$${x}_{r} = \frac{{L}_{32}}{2} - {x}_{2} {x}_{l} = - \frac{{L}_{32}}{2} - {x}_{2}$$

$${y}_{r} = \frac{{l}_{32}}{2} - {y}_{2} {y}_{l} = - \frac{{l}_{32}}{2} - {y}_{2}$$

$$h = {z}_{2} - {z}_{32}$$

$${r}_{1} = \sqrt{{x}_{l}^{2} + {y}_{l}^{2} + {h}^{2}} {r}_{2} = \sqrt{{x}_{r}^{2} + {y}_{l}^{2} + {h}^{2}}$$

$${r}_{3} = \sqrt{{x}_{r}^{2} + {y}_{r}^{2} + {h}^{2}} {r}_{4} = \sqrt{{x}_{l}^{2} + {y}_{r}^{2} + {h}^{2}}$$

$${q}_{2} = \sqrt{{y}_{l}^{2} + {h}^{2}} {q}_{4} = \sqrt{{y}_{r}^{2} + {h}^{2}}$$

where L₃₂ and l₃₂ are respectively the width measured along X and the length measured along Y of the top sensor 32, w is the angle between μ and X, and e is the angle between μ and Z. When the magnetic moment μ is aligned along Z, the field H₃₂ measured at the top sensor 32 becomes:

$${H}_{32} = \frac{h}{{q}_{2}^{2}} ( \frac{{x}_{l}}{{r}_{1}} - \frac{{x}_{r}}{{r}_{2}} ) + \frac{h}{{q}_{4}^{2}} ( \frac{{x}_{r}}{{r}_{3}} - \frac{{x}_{l}}{{r}_{4}} )$$

The field H₃₃ perceived at the second sensor 33 and emitted by the magnetic object 2 at the coordinates x₂, y₂ and z₂ can be obtained in the same way.

Knowing the passage height z₂ and fitting the magnetic fields H₃₂ and H₃₃, represented by the curves f_{TOP} and f_{BOTTOM} in FIG. 12, thus enables the magnetic moment μ to be determined accurately.

FIG. 1 shows an example of a reference curve R(z_{b}). In order for the results obtained from this curve to be accurate, it is preferred that the R curve takes the characteristics of biochip 3 into account. For this, it is generated, for example, by considering a calibration system. The calibration system comprises, for example, calibration magnetic field sensors and a calibration magnetic object. The calibration system is for example a model, for example a digital model. It may comprise calibration sensors which are, for example, models of the top and bottom sensors 32, 33. The calibration sensors are, for example, established from the relative positions of the top and bottom sensors 32, 33 with respect to each other and with respect to the microfluidic channel 31. They may also have calibration sensitivities, which are preferably equal to the sensitivities of the top and bottom sensors 32, 33. The calibration system may also comprise a calibration object. This object may have the same diameter as the magnetic object 2 which is desired to be characterised by means of the biochip 3 (this will be referred to as the target magnetic object). It may also have the same magnetic moment as the target magnetic object 2. It is advantageous, however, for the calibration magnetic object to have characteristics that are independent of the characteristics of the target magnetic objects 2. In this way, the reference curve can be used for different magnetic objects, whatever their magnetic moment or even diameter. The independence of the calibration object from the magnetic moment of the target magnetic object 2 is made possible by taking amplitude ratios rather than absolute values into account. The amplitude ratios are independent of the magnetic moments considered. The calibration magnetic object can thereby have a magnetic moment equal to 1.

In the example in FIG. 1, the reference curve R(z_{b}) corresponds to a ratio of the maximum dipole field emitted by the calibration object (having an arbitrary magnetic moment) and perceived at the calibration sensors. In this example, the calibration sensors are away by a distance of 26 μm, measured along the normal direction. This distance thus corresponds to a biochip 3 comprising a microfluidic channel 31 having a height hcan=20 μm (only this range of heights is illustrated in FIG. 1 since the target object 2 cannot penetrate the walls of the channel 31) and whose top and bottom sensors 32, 33 are away from the channel 31 by a distance equal to 5 μm and 1.7 μm respectively. In this example, the radius Ro of the target object 2 has been taken into account (although this is not compulsory), thereby limiting the range of passage heights z_{b} to be considered. Indeed, it extends between Ro and hcan-Ro.

The method 1 advantageously comprises a step of determining 10 the reference curve. According to one embodiment, the reference curve R(z_{b}) corresponds to a ratio of the maximum dipole field emitted by the calibration object (having an arbitrary magnetic moment) and perceived at the calibration sensors. The calculation of the fields perceived, as previously described (with reference to the fields H₃₂ and H₃₃), can be considered. It is herein the calculation of the field emitted by the calibration object with an arbitrary magnetic moment at the calibration sensors.

The calculation is made for different passage heights z of the calibration magnetic object between the calibration sensors. For each passage height z, the dipole magnetic field emitted by the calibration magnetic object and perceived by each calibration sensor is determined.

FIG. 12 shows, for example, a calculation of the maximum magnetic fields perceived by the calibration sensors (noted H₃₂ and H₃₃) and radiated by a calibration magnetic object with different magnetic moments. The magnetic moment is given as a function of a number of magnetic beads rather than in standard units (i.e. A·m²). For example, each magnetic bead has a magnetic moment equal to 1.56×10-14 A·m². The calculation is performed for a number of beads between 1 magnetic bead and 20 magnetic beads.

The maximum magnetic field perceived by a first calibration sensor is depicted as a solid line, while the maximum magnetic field perceived by a second calibration sensor is depicted as a dashed line. The maximum magnetic field perceived increases or decreases as a function of the passage height of the magnetic object.

Calculating the ratio of maximum magnetic fields as a function of passage height gives the reference curve, as illustrated in FIG. 1. The ratio calculated is constant as a function of the number of magnetic beads considered.

The calculation in FIG. 12 is of twofold interest in that it allows a theoretical Limit Of Detection (LOD) of electronic apparatuses to be taken into account. Below the theoretical limit of detection, it can be considered that the magnetic object will not be detected. Thus, the minimum magnetic moment (herein, the minimum number of beads) can be determined to enable the target object 2 to be properly characterised.

The ratio R of the amplitudes of the reference curve can be calculated from the magnetic fields perceived by the calibration sensors. When the sensitivities of the top and bottom sensors 32, 33 are identical, then the ratio of the amplitudes of the synchronised electrical signals is equal to the ratio R of the reference curve. However, the (real) top and bottom sensors 32, 33 may have different sensitivities. The ratio R then preferably takes the sensitivity of the top and bottom sensors 32, 33 into account. For this, the magnetic field perceived at each calibration sensor is multiplied by the sensitivity of the calibration sensor in question and preferably by the sensitivity of the top or bottom sensor 32, 33 it models.

The sensitivity factors of the top and bottom sensors 32, 33 are preferably linear and are preferably between 1/mT and 2%/mT.

When the magnetic object is a biological object marked, by means of magnetic beads, it is then advantageous for method 1 to comprise a step 14 for calculating the number of magnetic beads associated with said biological object. The calculation is made from the magnetic moment μ previously obtained and from the magnetic moment of a single magnetic bead. For example, Dynabeads (TM) brand magnetic beads have a magnetic moment μB=1.56×10⁻¹⁴ A·m². The magnetic moment of a single magnetic bead can be deduced from a measurement of the magnetic moment of an assembly of beads, for example by means of a Vibrating Sample Magnetometer (VSM). The number N of beads can then be obtained by N=p/μB.

The step of receiving 11 electrical signals from the sensors 32, 33 may comprise a first sub-step 11a of filtering the signals received. The filtering consists of a sliding mean for each signal. The sliding mean preferably has a window of about 0.1 ms.

The reception step 11 may also comprise a sub-step of identifying 11b characteristic signatures of the measurement of a magnetic object. [FIG. 7] shows an example of characteristic signatures. Each signature comprises a positive peak immediately followed by a negative peak. An inversion of the sensitivity of one of the sensors 32, 33 can invert this signature (which becomes a negative peak followed by a positive peak), as illustrated in [FIG. 8]. In such an event, the signal is preferentially inverted so that the signatures of the measurement of an object have a positive peak followed by a negative peak.

The characteristic signatures are first separated from the measurement noise. For this, a selection based on shape criteria can be implemented. For example, only peaks with an absolute amplitude greater than a threshold can be considered. Indeed, a minimum amplitude, both positive and negative, is expected from the peaks of a characteristic signature of a magnetic object 2. This amounts to considering voltage ranges, for example represented by the shaded areas around 0 V in FIG. 9, as comprising mainly measurement noise. The peaks considered have a prominence P_{TOP}, P_{BOTTOM}, measured outside the shaded areas, strictly greater than zero. The extent of the voltage ranges can be determined as a function of the noise density of the facility (measured without the targeted magnetic objects being introduced into the microfluidic channel 3). The noise may have a peak-to-peak voltage V_{b}, in which case the ignored ranges are equal [−V_{b}; V_{b}]. By virtue of this condition, signals generated by magnetic beads alone are ignored (especially as the dipole field they emit gives rise to signals whose amplitude is less than or equal to the LOD).

According to another example, compatible with the above example, only peaks with alternating polarities are considered. For example, a positive peak followed by a negative peak or a negative peak followed by a positive peak. It is expected that the time interval separating the extreme amplitudes of these two peaks is less than a predetermined duration. The predetermined duration varies as a function of the size of the magnetic object and its passage speed (the flow rate). For an object with a diameter of 10 μm in a flow of 8 cm/s, the predetermined time is between 100 us and 600 μs.

To facilitate identification of characteristic signatures, it is preferred that the signatures of several magnetic objects do not overlap. For this, it is preferable that the magnetic objects 2 are sufficiently diluted in the fluid matrix that allows them to circulate in the microfluidic channel 31.

Two marked biological objects give rise to signals that do not merge when they are separated by more than 30 μm. Considering a spacing z32-z33 between both sensors 32, 33, it is preferable that the dilution of the magnetic objects results in a spacing between two objects greater than or equal to 1.7×(z32−z33). Thus this method 1 is adapted to early diagnosis since the aggregation of biological objects satisfies these two criteria.

Reception step 11 may also comprise a sub-step 11c of identifying electrical signals synchronised between the characteristic signatures of the signal from the top sensor 32 and the characteristic signatures of the signal from the bottom sensor 33. Two synchronised characteristic signatures correspond to two measurements of a same magnetic object 2. The synchronisation of both characteristic signatures is thereby referred to as “coincidence”.

During the coincidence identification sub-step, a first instant is selected within a characteristic signature of the first signal and a second instant is selected within a characteristic signature of the second signal. The time offset t_{SYN} between these two times is measured. When the sensors 32, 33 are not aligned, the synchronised signatures may have an intrinsic time offset, which corresponds to the distance separating both sensors 32, 33 along the direction Y, multiplied by the flow rate in the channel 31. An acceptable range W_{SYN} of time offset t_{SYN} can therefore be determined. For example, for a flow of 8 cm/s, the range W_{SYN} of time offset t_{SYN} can be expected to extend over 100 μs. The characteristic signatures whose time offset is within this range are then said to be synchronised. For example, when the sensors 32, 33 are aligned (without any offset along Y), the intrinsic time offset is zero and the offset between both signatures is then within the 100 us range (i.e. with a relative difference of plus or minus 50 μs).

The first and second instants advantageously correspond to comparable characteristics for each signature. For example, when the first instant corresponds to the (positive) maximum of the characteristic signature of the first signal, then the second instant advantageously corresponds to the (positive) maximum of the characteristic signature of the second signal.

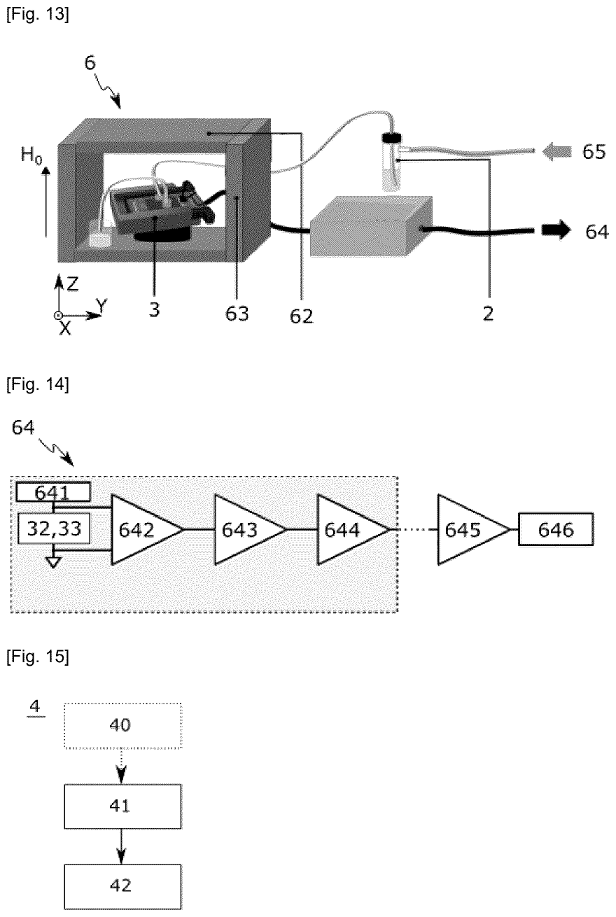

FIG. 13 schematically represents a device 6 for determining 1 the magnetic moment μ of magnetic objects 2. The device 6 comprises the biochip 3 as previously described. It also comprises complementary means 61, 62, 64, 65, adapted to implement the determination method 1 according to the invention.

The device 6 comprises, for example, a means 62 for applying a magnetic field H₀ to the biochip 3. The means 62 comprises, for example, a permanent magnet 62 configured to apply the field H₀ to the biochip 3. The field H₀ is preferably aligned along the direction Z. This field H0 makes it possible, for example, to align the magnetic moment μ of the magnetic objects along the direction Z (as illustrated in [FIG. 6]). In this way, the detection capacity of the sensors 32, 33 is maximal.

A soft iron 63 can be added to insulate the biochip from the external environment (especially the external magnetic field). This shielding 63 homogenises the magnetic field H₀ in which the biochip 3 is soaked and also improves the signal-to-noise ratio of the sensors 32, 33.

The magnetic objects 2 to be characterised are preferably mixed in a fluid matrix. In this way, the fluid matrix and the magnetic objects 2 can be circulated in the microfluidic channel 31 of the biochip 3. The device 6 advantageously comprises a system 65 for controlling flow rate of the fluid matrix in which the magnetic objects 2 are soaked.

The device 6 can also comprise an acquisition chain 64 responsible for polarising the sensors 32, 33 (for example in current) and receiving the electrical signals from the sensors 32, 33.

FIG. 14 shows an example of an acquisition chain 64. It especially comprises a current source 641 responsible for polarising current to the sensors 32, 33. A voltmeter 642 is used to measure the voltage across each sensor 32, 33. The voltmeter 642 advantageously comprises an amplification stage, for example of +60 dB, in order to provide usable voltage values. The voltmeter may also comprise a high-pass filter in order to eliminate very low frequency noise, for example below 150 Hz. The acquisition chain 64 may also comprise an additional amplification stage 643. It may also include a low-pass filter 644, responsible for ejecting very high frequencies, for example above 37 kHz.

The chain 64 may also comprise a digital converter 645 and a controller 646, responsible for processing electrical signals from the sensors 32, 33. The sampling frequency of the converter 645 partly depends on the flow rate. For example, for a flow rate, the sampling frequency is preferably greater than or equal to 200 KHz.

The controller 646 is used, for example, to perform calculation steps 12, 13 and 14 of method 1.

Determining 1 the magnetic moment μ of the magnetic objects 2 passing into the microfluidic channel 31 can be made in “real time”, i.e. simultaneously with the passage of the magnetic objects 2 between the sensors, or at a later time. In the latter case, the TOP, BOTTOM signals measured by the sensors 32, 33 are recorded in files and processed later.

FIG. 15 schematically illustrates a method 4 for counting biological objects 5 implementing biochip 3 and the method for determining a magnetic moment, as previously described.

The biological objects 5 are, for example, biological targets of various sizes, such as cells, bacteria or even viruses. Examples include cancer cells or pathogenic bacteria such as salmonella, Yersinia enterocolitica or even Legionella pneumophila.

With the exception of magnetosomes, which have an intrinsic magnetic core, biological objects 5 are not magnetic. It is therefore necessary to perform marking thereof which assign a magnetic moment thereto. In this way, they can be counted using a method based on a magnetic moment measurement.

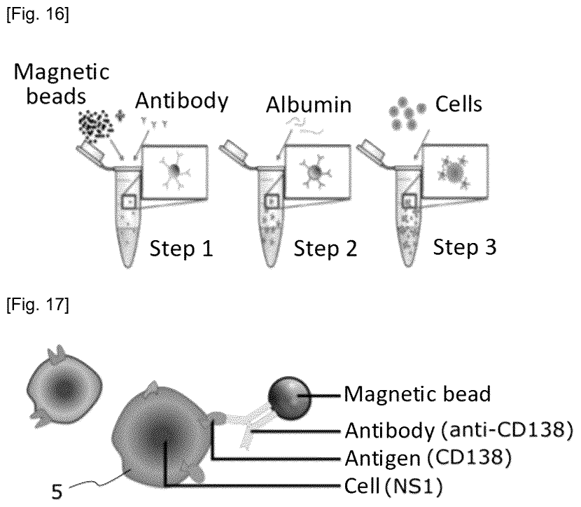

FIG. 16 schematically illustrates a biological object marking step. For this, the counting method 4 first comprises a step of marking biological objects 5. To this end, the biological objects 5, herein cells, are mixed with a matrix comprising magnetic beads. The magnetic beads are able to mark the biological objects 5. They are associated, for example, with antibodies able to couple with antigens carried by the biological objects 5.

Among magnetic beads (ranging in size from 200 nm to 1 μm, for example), three types of surface functionality exist. The sulphonic ester and carboxylic acid groups chemically react with antibodies to create covalent bonds, while streptavidin beads are functionalised via biotin-streptavidin interaction by antibodies previously covalently coupled to biotin.

For example, in a first step, magnetic beads ranging in size from 200 nm to 1 μm and antibodies are mixed in a neutral solution. The protocol is specific for each batch of beads studied. The magnetic beads are, for example, micrometric beads made up of nanometric iron oxide (maghemite or magnetite) cores wrapped in a polymer layer. The antibodies bind to the polymer layer of the magnetic beads.

The antibody-functionalised beads are added to the matrix containing the biological objects 5. They are added in excess in order to promote probability of encountering the biological objects.

Some of the antibodies will associate with antigens carried by each cell 5. In this way, the cells 5 are said to be “marked”, i.e. functionalised by means of magnetic beads. The cells 5 marked carry, for example, between 50 and 100 magnetic beads with a diameter of 1 μm. Despite the precautions taken, some of the cells 5, in the order of 7%, may remain non-marked. Moreover, the larger the cells 5 marked are, the more likely they are to carry a high number of magnetic beads, which may make them easier to count.

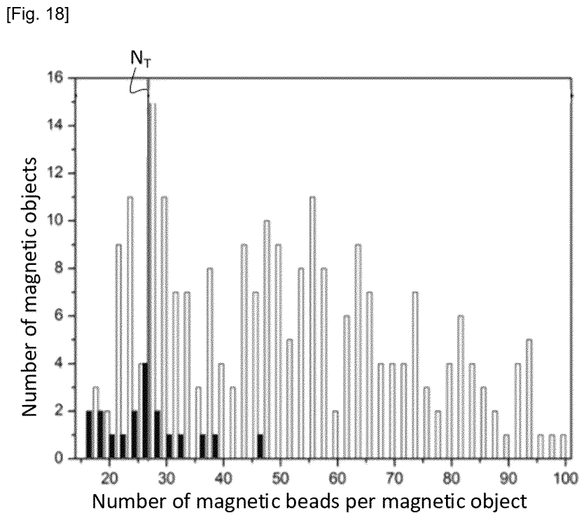

FIG. 17 shows an example of a biological object 5 marked, herein a cell comprising antigens, one of which is associated with an antibody. A magnetic bead is bound to the antibody in question. By virtue of the magnetic bead, the biological object 5 marked is carrying a magnetic moment u.

The counting method of [FIG. 15] also comprises a step of determining the magnetic moment μ of magnetic objects passing between sensors 32, 33. During this step, the matrix comprising the biological objects 5 marked circulates in the microfluidic channel 31 of the biochip 3 (as previously described). Electrical signals from the sensors 32, 33 are acquired as the biological objects 5 circulate in the channel 31. For each coincidence, as previously described, a magnetic moment μ is calculated.

FIG. 18 illustrates, for example, a distribution diagram of the magnetic objects 2 detected as a function of the number of magnetic beads possessed by the magnetic objects detected. The abscissa in the figure indicates a number of magnetic beads per characterised magnetic object rather than the value of the magnetic moment. The number of magnetic beads is determined as the ratio of the magnetic moment of the detected object and the individual magnetic moment of the magnetic bead (determined in a preliminary manner).

The white bars in [FIG. 18] correspond to counting made in a specific sample comprising marked biological objects of interest in a complex matrix (culture medium). The black bars, on the other hand, correspond to a so-called “negative control” sample containing the same complex matrix (with the same number of functionalised magnetic beads) but without any marked biological object (it is herein the by-product of step 2 in FIG. 17).

In the same case (black bars), magnetic objects are detected and show a magnetic moment. However, these are not target biological objects (as they are not present in the matrix to make these specific measurements). They are magnetic aggregates that can be confused with biological objects marked. They should therefore be distinguished from biological objects 5 marked. These magnetic aggregates comprise between two and twenty-five magnetic beads per aggregate. They can only be detected from four magnetic beads when they pass through the centre and from twelve beads over the whole channel. Below four magnetic beads, the aggregates emit a dipole field which is generally below the limit of detection (referred to as “LOD”).

In order to avoid counting magnetic aggregates, a threshold magnetic moment is considered. In the example of FIG. 18, this corresponds to a threshold number of magnetic beads, equal, in the example provided, to twenty-five beads.

Consequently, the counting method 4 can count each magnetic object for a marked biological object if the magnetic moment of the magnetic object (and therefore the number of magnetic beads it carries) is greater than the threshold magnetic moment (i.e. greater than the threshold number of magnetic beads NT). In this way, the magnetic aggregates are not counted.

1. A method for determining the magnetic moment of a magnetic object by means of a biochip, the magnetic object being a biological object marked by means of magnetic beads or a magnetosome or an aggregate of magnetic beads, the biochip comprising: a microfluidic channel extending in a plane and having a height, measured along a direction referred to as the “normal direction” perpendicular to the plane; and two magnetic field sensors, disposed on either side of the microfluidic channel, the method comprising the following steps of:
receiving two synchronised electrical signals from the magnetic field sensors respectively, corresponding to the passage of the magnetic object into the microfluidic channel at each magnetic field sensor;
calculating the passage height of the magnetic object, measured along the normal direction, from the ratio of the amplitudes of the synchronised electrical signals and by means of a reference curve relating the passage height of a calibration magnetic object in the microfluidic channel to a ratio of the amplitudes of the calibration electrical signals corresponding to the passage of the calibration magnetic object at said passage height, said ratio of the amplitudes of the calibration electrical signals non-linearly increasing as the passage height of the calibration magnetic object increases until it reaches a maximum value, the ratio of the amplitudes of the synchronised electrical signals being equal to the ratio of the amplitudes of the calibration electrical signals,
calculating the magnetic moment of the magnetic object from the passage height determined and one of the synchronised electrical signals.

2. The method according to the preceding claim 1, wherein the reference curve is determined from the calibration magnetic object having a calibration magnetic moment independent of the magnetic moment of the magnetic object.

3. The method according to claim 1, wherein the electrical signal from each magnetic field sensor is equal to the dipole field emitted by the magnetic object multiplied by a sensitivity factor of the magnetic field sensor.

4. The method according to the preceding claim 3, wherein each calibration electrical signal is equal to the dipole field emitted by the calibration magnetic object at a calibration magnetic field sensor multiplied by a calibration sensitivity factor, the calibration sensitivity factors being equal to the sensitivity factors of both magnetic field sensors.

5. The method according to claim 1 one of the preceding claims, comprising a step (10) of determining the reference curve comprising the following sub-steps of:
for different passage heights of the calibration magnetic object between two calibration magnetic field sensors:
determining the dipole magnetic field emitted by the calibration magnetic object and perceived by one of the calibration magnetic field sensors;
determining the dipole magnetic field emitted by the calibration magnetic object and perceived by the other calibration magnetic field sensor;
calculating the ratio of the amplitudes of the dipole magnetic fields perceived by the calibration magnetic field sensors as a function of the different passage heights of the calibration magnetic object.

6. The method according to claim 1, wherein the reception step also comprises a sub-step of identifying synchronised electrical signals comprising measuring a time difference between characteristic signatures of both electrical signals, each characteristic signature corresponding to the measurement of the magnetic object by one of the magnetic field sensors, synchronisation being identified when the time difference is within a predetermined time range.

7. The method according to claim 5 one of the two preceding claims, wherein the reception step comprises a sub-step of identifying characteristic signatures in each of the electrical signals from shape criteria of the electrical signals.

8. The method according to claim 1, wherein the magnetic object is a biological object marked by means of magnetic beads, said method also comprising a step of calculating the number of magnetic beads associated with the biological object marked from the magnetic moment calculated and the magnetic moment of a single magnetic bead.

9. The method according to claim 1 one of the preceding claims, wherein the magnetic field sensors are magnetoresistive sensors.

10. A method for counting biological objects, comprising the following steps of:
marking the biological objects by mixing them with a liquid matrix comprising magnetic beads able to attach to receptors carried by each biological object;
circulating the liquid matrix comprising the biological objects marked in the microfluidic channel of the biochip and determining the magnetic moment of magnetic objects passing between the magnetic field sensors by means of the method according to claim 1, each magnetic object counting as a biological object marked when the magnetic moment associated therewith is greater than a threshold magnetic moment.

11. The counting method according to the preceding claim 10, comprising the step of determining the threshold magnetic moment, said step comprising the following sub-steps of:
circulating the liquid matrix without biological objects, referred to as the “negative control”, in the microfluidic channel of the biochip and determining the magnetic moment of magnetic objects passing between the magnetic field sensors by means of the method for determining the magnetic moment according to the invention; and
setting the threshold magnetic moment from a statistical distribution of the magnetic moments determined.

12. A device for determining the magnetic moment of a magnetic object,
the magnetic object being a biological object marked by means of magnetic beads or a magnetosome or an aggregate of magnetic beads, comprising:
a biochip comprising: a microfluidic channel extending in a plane and having a height, measured along a direction referred to as the “normal direction” perpendicular to the plane; and two magnetic field sensors, disposed on either side of the microfluidic channel,
a magnet configured to apply a magnetic field at the biochip,
a system for controlling flow rate of a fluid matrix configured to circulate a liquid matrix comprising the biological objects marked in the microfluidic channel of the biochip,
an acquisition chain responsible for polarising the sensors and receiving both synchronised electrical signals from said sensors, corresponding to the passage of the magnetic object into the microfluidic channel at each magnetic field sensor; and comprising a controller configured to:
calculate the passage height of the magnetic object, measured along the normal direction, from the ratio of the amplitudes of the synchronised electrical signals and by means of a reference curve relating the passage height of a calibration magnetic object in the microfluidic channel to a ratio of the amplitudes of the calibration electrical signals corresponding to the passage of the calibration magnetic object at said passage height, said ratio of the amplitudes of the calibration electrical signals non-linearly increasing as the passage height of the calibration magnetic object increases until it reaches a maximum value, the ratio of the amplitudes of the synchronised electrical signals being equal to the ratio of the amplitudes of the calibration electrical signals, and
calculate the magnetic moment of the magnetic object from the passage height determined and one of the synchronised electrical signals.

13. A system for counting biological objects,
comprising:
means for marking the biological objects by mixing them with a liquid matrix comprising magnetic beads able to attach to receptors carried by each biological object;
the device for determining the magnetic moment of a magnetic object according to claim 12, wherein the controller of the acquisition chain is further configured to count a biological object marked when the magnetic moment associated therewith is greater than a threshold magnetic moment.

14. A non-transitory computer readable medium comprising instructions which cause the device according to claim 12 to execute the steps of the determination method according to claim 1.

15. A non-transitory computer readable medium comprising instructions which cause the counting system according to claim 13 to execute the steps of a counting method.
